SEQUENCE LISTING (14 sequences)

SEQ ID NO: 1 - DNA, 2592 bp, Lemna paucicostata
atggccggttttctccaaaaggtaaccgataatcttttgcacaaggcggccaagatctctgggactgttgtgttagtaaagagcaatgtcgtcgggttcaacgatttcgctgactcgttcttggacggtctgcacgagcttctgggcagcggcatcaccttccagctcgtcagcgccaccgtcggcgatccaaagaatgggaacaagggcaagctaggaaagccggcgctgcttgagaaatggatcacgaccgggacctttctcgcggcgggagactcaggcttcaaggtgaacttcgagtgggacgagcagctcggcgttcccggcgcggtcatcgtgaaaaacaaccaccacggcgagttcttcctcaagagcttgactctggatggcgcccctggcccccgaagccgcatccacttcgactgcaactcctgggtttatccttatagcacctacaactacgatcgggttttcttcgctaacgacacgtaccttccgggagaaatgccggagccgctgaaagcctacagggcggcggagctggtgaatctgagaggtgacggggtgacccgcgagctgaaggagggagatcgaatctacgcttacgacttgtacaacgacctcggcgatccggacagcggcaaagagttagcccggccgatcctcggcggctcggaagagtatccctaccctcgccgggcccggacaggccggaagttgacgaagactgatccaaagtcggagcaaaggcttccccttgtgtttagcctgaacgtgtacgtccccagagacgagcgctttgggcatctgaagatgtccgatttcttggcctactccctaaaggctctggctcaggggttggtgccggcgttagatgctgcgaccgacataactcctttcgagttcgataccttccaagacgtcttgaatctctacgagggaggcatcaatttgcccactactcccgctttagaaaacttcaagaaacagatcccattccccctcgtaaaggagcttttcaggtccgacggcgagaacctgtttcgccttcccactccctccgttatcaaagctgacaagttcgcctggaggactgacgaggagttcggaagggagatgctcgccggagtcaatcccgtttgcattagactcttaaagaaatttcccccagtcagcagtcttgacccgagcatttaccgcaaccagaacagcaccatgattgctgatcagctcgagaagaacatgaacgggctctccgtcgaagaggccttaaagcagaagaagctcttcatcttggaccatcacgatagtctgatgccctacctggagcgcataaacacctttaacaaaatctacgcctccagaaccgtgttactgcttcaggacgatggaaccctaaaaccgctagccattgagcttagcttacccgtcaaaggcgagaaaggagctgtcagcaaggtatacacgccggcagagcatggcgtcgagggcgccgtctggcagctcgccaaggcctacgttgccgtcaatgattccggcgttcaccaacttatcagccactggctgaacacccacgccactatagagcctttctccatcgctctcaacaggcagctcagcgtggtgcacccaatatacaagctccttcatccccacttccgtgacaccatgaacatcaacgccttcgccagacaaatccttattaatgccggcggaatcctcgagatgacggtcttcccagggaagtacgccatggagatgtcctccgtggtgtacaaaggatggaaactcacggaccaagcccttccggtggacctcctcaacaggggtgtggctgaaaaagatccatcctcgggagagcttcggcttttgatcgaggattatccgtacgcggtcgacgggctagacgtctggaatgccattgaggaatgggtcaaggagtactgcgcaatctactacccctcggacaagaccctgcaggatgacaccgaggtccaagcctggtggaaggaggtgcgtgaggtgggacacggcgacaagaaggatgaaacatggtggccggccatggagaccgtggctgagctgacccagacttgctccacgatcatctgggtggcctccgccctccacgccgccgtcaactttgcgcagtacccatacgctggttatctgcccaaccgtccgactattagtcgacggttcatgcccgaaccgggcacgccggagtacgaggagctagaaaccgaccccgaccgggcattcctcaaaacgattaccagtcagctgcagacccttatcggtgtttctctgatcgagatcctctccagacactcgtctgatgaaatctatctagggcagagagttagcgccgagtggacttcggacgccaaggcgctggctgccttcaaggctttcggtgaaaagctgatcaccatcgaaaacaagatcatcgagatgaacgtggactccagcctgaagaatcgcaacggccccgtcaaggtcccgtacacttttctctaccccaacaccaccgattatacccgtgtgggtgggctgaccggtcgtgggatccccaacagcatctccatttga

SEQ ID NO: 2 - protein, 863 aa, Lemna paucicostata
MAGFLQKVTDNLLHKAAKISGTVVLVKSNVVGFNDFADSFLDGLHELLGSGITFQLVSATVGDPKNGNKGKLGKPALLEKWITTGTFLAAGDSGFKVNFEWDEQLGVPGAVIVKNNHHGEFFLKSLTLDGAPGPRSRIHFDCNSWVYPYSTYNYDRVFFANDTYLPGEMPEPLKAYRAAELVNLRGDGVTRELKEGDRIYAYDLYNDLGDPDSGKELARPILGGSEEYPYPRRARTGRKLTKTDPKSEQRLPLVFSLNVYVPRDERFGHLKMSDFLAYSLKALAQGLVPALDAATDITPFEFDTFQDVLNLYEGGINLPTTPALENFKKQIPFPLVKELFRSDGENLFRLPTPSVIKADKFAWRTDEEFGREMLAGVNPVCIRLLKKFPPVSSLDPSIYRNQNSTMIADQLEKNMNGLSVEEALKQKKLFILDHHDSLMPYLERINTFNKIYASRTVLLLQDDGTLKPLAIELSLPVKGEKGAVSKVYTPAEHGVEGAVWQLAKAYVAVNDSGVHQLISHWLNTHATIEPFSIALNRQLSVVHPIYKLLHPHFRDTMNINAFARQILINAGGILEMTVFPGKYAMEMSSVVYKGWKLTDQALPVDLLNRGVAEKDPSSGELRLLIEDYPYAVDGLDVWNAIEEWVKEYCAIYYPSDKTLQDDTEVQAWWKEVREVGHGDKKDETWWPAMETVAELTQTCSTIIWVASALHAAVNFAQYPYAGYLPNRPTISRRFMPEPGTPEYEELETDPDRAFLKTITSQLQTLIGVSLIEILSRHSSDEIYLGQRVSAEWTSDAKALAAFKAFGEKLITIENKIIEMNVDSSLKNRNGPVKVPYTFLYPNTTDYTRVGGLTGRGIPNSISI

SEQ ID NO: 3 - DNA, 2610 bp, Lemna paucicostata
atggatgcttttatccacaaggtaactgatgtttttacgcacaaggaggagaacaaggtcgccaagatctccgggactgtggtgatagtgaagagcaacgtcgtcgggttcaacgacttcgttgactcgttcctcgacggcctgcacgagctcgtcggcagcggcatcaccttccagctaatcagcgccaccgtcggcgatccaaagaatgggaataaaggcaagctaggaaagccggcgcagttggagagatggatcacgactgggacctttctcgctgctggagactcgggtttcaaattgaccttcgagtgggatgagcagctcggctttcccggagccgtcatcgtgaaaaacaaccaccacggcgagttcttcctcaaaagcttcactctggacggcgtccctggcaagaaaacccgcattcacttcgactgtaactcttgggtctatccttatagcacctacaactacgatcgggtcttcttcgcgaacgacacattcctacccggaaacatgccggagccgctgaaagcctacagggcggcggagctggcaaatctgagaggcgacggagtagccccccgcgagttgaaggaaggagatcgaatttacgcttacgacgtctacaacgacctcggcgatccggacagcggcgaggagttggtccggccgattctcggcggctcgaaagagtatccctaccctcgaagagcccggactggtcggaagatgaccaagacagatcccaagtcggagacgaggcttccacttttgttcagtttgaacgtgtatgtccccagagacgagcgctttgggcatctgaaaatgtccgatttcttgggctactcgctcaaggctctggctcaggggctggtgcccgtgttaaatgaggcggccgacttaactcctttcgagtttgatacattccaggacgtattgaatctctacgaggggggcatagatctgcccgacagtcccgccttagaaaacgtcaggaaacagatcccctttgcactcataaaggagcttttcaggtccgacggcgagaacctgtttcgccttccgactcccgccgttatcaaagctgacaagttcgcctggaggactgatgaggagttcggaagggaaatgcttgccggggtcaaccctgtctgcattagactcttaacgaaatttcccccggtcagcagccttgaccctaacacttacggcgaccacaacagtaccatgaccgctgatcagctcgagaagaacatgaacgggctctccgtcgaagaggcccttaataaaaagaagctcttcatcttggaccatcacgacagtctgatgccctacctagagcgcataaacacctttaacaagatctacgcctccagaaccgtgttactgcttcaggacgatggaaccctaaaaccgctagccattgagctgagcttacccatcaagggtcagaagggagccgtcagcaaggtatacacgcctgcggagcatggcgtcgaggctgccgtgtggcagctcgcgaaggccttcgctgcggtgaacgattccggcgttcaccagcttatcagtcactggctgaacacccacgccaccattgagcctttcgccatcgctctcaacaggcagctcagcgtagtgcacccaatatacaagctccttcatccccacttccgcgacaccatgaacatcaacgccttcgccagacagatcctcattaacgccggcggaatcctggaaatgacggtcttcccaggcaagtatgccttggagatgtcctccgtagtgtacaaaggctggaaactcatcgaccacgctcttcccgttgacctcctcaacaggggtgtggctgaaaatgatccatccgcccccggaaagctccgtttgttgatcgaggactacccgtacgcggtcgacgggcttgctgtctggtctgccattgagatctgggtgaaggagtactgcgcaatctactacccctcggacaagatcctgcaggatgacaccgagctccaagcctggtggaaggaggtgcgggaggtgggacacggcgacaagaaggacgaaacgtggtggccgaccatgaagaccgtggccgagctgacccaaacttgctgcacgatcatctgggtggcctcggccctccacgccgccgtcaactttgggcagtacccgtacgccggctacctccccaaccgtcccaccgtaagccggagattcatgcccgaacctggcacgcctgagtacgaagagctgaaaaccgaccccgacagggcgttcctcaaaacgattacaagccagctgcagaccacaattggcgtgtctctcatcgagatcctctcaaggcactcttccgacgaggtctatctcggtcagagaatcagcgccgagtggactacggacgagaaggcgctggccgccttcaaggcgtttggtgagagactgatcacgatcgagaaccagatcatcgagatgaaccatgagcctaaactgaagaaccgtaacgggcccgtgaagcttccgtacacactgctttaccccaacacatcggattacacccgcgtgggtgggctcaccggtcgcggcatccccaatagcgtctccatttga

SEQ ID NO: 4 - protein, 869 aa, Lemna paucicostata
MDAFIHKVTDVFTHKEENKVAKISGTVVIVKSNVVGFNDFVDSFLDGLHELVGSGITFQLISATVGDPKNGNKGKLGKPAQLERWITTGTFLAAGDSGFKLTFEWDEQLGFPGAVIVKNNHHGEFFLKSFTLDGVPGKKTRIHFDCNSWVYPYSTYNYDRVFFANDTFLPGNMPEPLKAYRAAELANLRGDGVAPRELKEGDRIYAYDVYNDLGDPDSGEELVRPILGGSKEYPYPRRARTGRKMTKTDPKSETRLPLLFSLNVYVPRDERFGHLKMSDFLGYSLKALAQGLVPVLNEAADLTPFEFDTFQDVLNLYEGGIDLPDSPALENVRKQIPFALIKELFRSDGENLFRLPTPAVIKADKFAWRTDEEFGREMLAGVNPVCIRLLTKFPPVSSLDPNTYGDHNSTMTADQLEKNMNGLSVEEALNKKKLFILDHHDSLMPYLERINTFNKIYASRTVLLLQDDGTLKPLAIELSLPIKGQKGAVSKVYTPAEHGVEAAVWQLAKAFAAVNDSGVHQLISHWLNTHATIEPFAIALNRQLSVVHPIYKLLHPHFRDTMNINAFARQILINAGGILEMTVFPGKYALEMSSVVYKGWKLIDHALPVDLLNRGVAENDPSAPGKLRLLIEDYPYAVDGLAVWSAIEIWVKEYCAIYYPSDKILQDDTELQAWWKEVREVGHGDKKDETWWPTMKTVAELTQTCCTIIWVASALHAAVNFGQYPYAGYLPNRPTVSRRFMPEPGTPEYEELKTDPDRAFLKTITSQLQTTIGVSLIEILSRHSSDEVYLGQRISAEWTTDEKALAAFKAFGERLITIENQIIEMNHEPKLKNRNGPVKLPYTLLYPNTSDYTRVGGLTGRGIPNSVSI

SEQ ID NO: 5 - DNA, 2592 bp, Lemna paucicostata
atggccgattttctccaaaaggtaaccaataatcttttacacaaggcggccaagatctctgggactgttgtgttagtaaagagcaatgtcatcgggttcaacgatttcggtgactcgttcttggacggtctgcacgaacttctgggcagcggcatcaccttccagctcgtcagcgccaccgtcggcgatccaaataatgggaacaagggcaagctaggaaagccggccctgcttgagaaatggatcacgaccgggacctttctcgtggcgggagactctggcttcaaggtgaacttcgagtgggacgagcagctcgggattcccggcgcagtcatcgtgaaaaacaaccaccacggcgagttcttcctcaagagcttcactctggatggcgtccctggccccagaagccgcatccacttcgattgcaactcctgggtttatccctatagcacctacaactacgatagggttttcttcgctaacgacacgtaccttccgggagaaacgccggagccgctgaaagcctacagggccgcggagctggtgaatctgagaggtgacggggtgacccgcgagctgaaggagggagatcgaatctacgcttacgactactacaacgacctcggcgatccggacagcggcaaagagttagcccggccaatcctcggcggctcgaaagagtatccctaccctcgccgggcccggacaggccggaagttgacgaagactgatccaaagtcggagcaaaggcttccgctggtgtttagcctgaacgtgtacgtccccagagacgagcgctttgggcatctgaagatgtccgatttcttggcctactccctcaaggctctggctcaggggttggtgccggcgttagatgctgcggccgacataactcctttcgagttcgataccttccaagacgtcttgaatctctacgagggaggcatcaatttgcccactactcccgcttcagaaaacttcaagaaacagatcccattccccctcgtaaaggagcttttcaggtccgacggcgagaacgtgtttcgccttccgactccctccgttatcaaagctgacaagttcgcctggaggactgacgaggagttcggaagggagatgcttgccggagtcaaccccgtttgcattagactcttaacgaaatttcccccagtcagcagtcttgacccgagcatttacggcaaccagaacagcaccatgattgctgatcagctcgagaagaacatgaacgggctctccgtcgaagaggccttaaagcagaagaagctcttcatcttggaccatcacgatagtctgatgccctacctggagcgcataaacacctttaacaaaatctacgcctccagaaccgtgttactgcttcgggacgatggaaccctaaaaccgctagccattgagcttagcttacccatcaaaggcgagaaaggagctgtcagcaaggtatacacgccggcagagcatggcgtcgagggcgccgtctggcagctcgccaaggcctacgttgccgtcaatgattccggcgttcaccaacttataagccactggctgaacacccacgccactatagagcctttctccatcgctctcaacaggcagctcagcgtggtgcacccaatatacaagctcctccatccccacttccgtgacaccatgaacatcaacgccttcgccagacaaatccttattaatgccggcggaatcctcgagatgacggtcttcccaggcaagtacgccatggagatgtcctccgtggtgtacaaaggatggaaactcacggaccaagccctaccggtggacctcctcaacaggggtgtggctgaaaaagatccatcctcgggagagcttcggcttttgatcgaggactatccgtacgcggtcgacgggctagacgtctggaatgccattgaggaatgggtcaaggagtactgcgcaatctactacccctcggacaagaccctgctggatgacaccgagctccaagcctggtggaaggaggtgcgtgaggtgggacacggtgacaagaaggatgaaacatggtggccggccatggagaccgtggccgagctgacccagacttgctgcacgatcatctgggtggcctccgccctccacgccgccgtcaactttgcgcagtacccatacgccggttacctgcccaaccgtccgactattagtcgacggttcatgccagaaccgggcacgccggagtacgaggagctagaaaccgaccccgaccgggcattcctcaaaacgattaccagtcagctgcagacccttatcggtgtttctctgatcgagatcctctccagacactcgtctgatgaaatctatctagggcagagagttagcgccgagtggacttcggacgccaaggcgctggctgccttcaaggctttcggtgaaaagctgatcaccatcgaaaacaagatcatccagatgaacgtggactccagcctgaagaatcgcaacggccccgtcaaggtcccgtacacttttctctaccccaacaccaccgattatacccgtgtgggtgggctgaccggtcgtggaatccccaacagcatctccatttga

SEQ ID NO: 6 - protein, 863 aa, Lemna paucicostata
MADFLQKVTNNLLHKAAKISGTVVLVKSNVIGFNDFGDSFLDGLHELLGSGITFQLVSATVGDPNNGNKGKLGKPALLEKWITTGTFLVAGDSGFKVNFEWDEQLGIPGAVIVKNNHHGEFFLKSFTLDGVPGPRSRIHFDCNSWVYPYSTYNYDRVFFANDTYLPGETPEPLKAYRAAELVNLRGDGVTRELKEGDRIYAYDYYNDLGDPDSGKELARPILGGSKEYPYPRRARTGRKLTKTDPKSEQRLPLVFSLNVYVPRDERFGHLKMSDFLAYSLKALAQGLVPALDAAADITPFEFDTFQDVLNLYEGGINLPTTPASENFKKQIPFPLVKELFRSDGENVFRLPTPSVIKADKFAWRTDEEFGREMLAGVNPVCIRLLTKFPPVSSLDPSIYGNQNSTMIADQLEKNMNGLSVEEALKQKKLFILDHHDSLMPYLERINTFNKIYASRTVLLLRDDGTLKPLAIELSLPIKGEKGAVSKVYTPAEHGVEGAVWQLAKAYVAVNDSGVHQLISHWLNTHATIEPFSIALNRQLSVVHPIYKLLHPHFRDTMNINAFARQILINAGGILEMTVFPGKYAMEMSSVVYKGWKLTDQALPVDLLNRGVAEKDPSSGELRLLIEDYPYAVDGLDVWNAIEEWVKEYCAIYYPSDKTLLDDTELQAWWKEVREVGHGDKKDETWWPAMETVAELTQTCCTIIWVASALHAAVNFAQYPYAGYLPNRPTISRRFMPEPGTPEYEELETDPDRAFLKTITSQLQTLIGVSLIEILSRHSSDEIYLGQRVSAEWTSDAKALAAFKAFGEKLITIENKIIQMNVDSSLKNRNGPVKVPYTFLYPNTTDYTRVGGLTGRGIPNSISI

SEQ ID NO: 7 - DNA, 24 bp, Artificial Sequence
  Synthesized forward degenerate primer for LOX
gcntggmgnacngaygargartty

SEQ ID NO: 8 - DNA, 23 bp, Artificial Sequence
  Synthesized reverse degenerate primer for LOX
gcrtanggrtaytgnccraartt

SEQ ID NO: 9 - DNA, 18 bp, Artificial Sequence
  Synthesized 3′-race primer for LOX derived from SH strain
agctcttcatcttggacc

SEQ ID NO: 10 - DNA, 22 bp, Artificial Sequence
  Synthesized 3′-race primer for LOX-1 derived from 441 strain
aagcttcttcatccccacttcc

SEQ ID NO: 11 - DNA, 22 bp, Artificial Sequence
  Synthesized 3′-race primer for LOX-2 derived from 441 strain
aagctccttcatccccacttcc

SEQ ID NO: 12 - DNA, 18 bp, Artificial Sequence
  Synthesized 5′-race primer for LOX derived from SH strain
tttcatccttcttgtcgc

SEQ ID NO: 13 - DNA, 19 bp, Artificial Sequence
  Synthesized 5′-race primer for LOX-1 derived from 441 strain
gcttgtatattgggtgcac

SEQ ID NO: 14 - DNA, 23 bp, Artificial Sequence
  Synthesized 5′-race primer for LOX-2 derived from 441 strain
aagcagtaacacggttctggagg

(19) United States
(12) Patent Application Publication
(10) Pub. No.: US 2013/0224829 A1
(43) Pub. Date: Aug. 29, 2013

(54) NOVEL LIPOXYGENASE DERIVED FROM LEMNA PAUCICOSTATA

(71) Applicants: Mineyuki Yokoyama, Yokohama-shi (JP); Kazuteru Takagi, Yokohama-shi (JP); Sari Kamichi, Yokohama-shi (JP)

(72) Inventors: Mineyuki Yokoyama, Yokohama-shi (JP); Kazuteru Takagi, Yokohama-shi (JP); Sari Kamichi, Yokohama-shi (JP)

(21) Appl. No.: 13/583,950

(22) PCT Filed: Mar. 11, 2011
(86) PCT No.: PCT/JP2011/055834

§ 371 Date: Sep. 11, 2012

(30) Foreign Application Priority Data
Mar. 11, 2010 (JP) 2010-055099

(51) Int. Cl. C12N 9/02; C12N 5/10; C12N 1/00; C12N 15/53; C12N 15/63

(52) U.S. Cl. 435/189; 536/23.2; 435/320.1; 435/243; 435/325; 435/419

(57) ABSTRACT

A highly active novely lypoxygenase cDNA derived from Lemna paucicostata SH strain and Lemna paucicostata 441 strain and a protein encoded by the cDNA are provided.

### TECHNICAL FIELD

The present invention relates to a novel highly active form of position 9 product specific lipoxygenase gene derived from Lemna paucicostata, a method of producing highly active form of position 9 product specific lipoxygenase by using the lipoxygenase gene, and an expression product of said lipoxygenase gene.

### BACKGROUND ART

Lipoxygenase is an enzyme having an activity to directly introduce molecular oxygen into unsaturated fatty acid having 1,4-pentadiene structure, such as linoleic acid, linolenic acid (carbon number 18), or arachidonic acid (carbon number 20) to produce hydroperoxyl group (—OOH), and is known to be present in plants, animals, and eukaryotic microorganisms, such as fungis, yeast, and so on (Uchiyama Mitsuru, et al., lipid peroxide and biological body, 23-26, 1985, Japan Scientific Societies Press). In animals, since this enzyme relates to the process of producing prostaglandin from arachidonic acid, research is most ahead (Yamamoto Shouzo, Protein Nucleic acid Enzyme, 44, 1132-1138, 1999). Lipoxygenase in plants is known to be classified to an enzyme which shows specificity to two positions, position 9 and position 13, as using a unsaturated fatty acid with carbon number of 18, such as α-linolenic acid, linoleic acid, as a substrate (for example, Uchiyama Mitsuru, et al., lipid peroxide and biological body, 23-26, 1985, Japan Scientific Societies Press, and Grechkin et al., Eur. J. Biochem., 199, 451-457, 1991). Position 9 and position 13 specific lipoxygenase was known to be present in potato, soybean, tomato, pea, barley, wheat, corn, cucumber, and so on, and some cDNAs have been isolated. However, position 13 product specific lipoxygenase, which relates to a metabolic pathway of jasmonic acid, which attracts attention as a plant hormone, is mainly investigated, and position 9 product specific lipoxygenase is relatively less investigated. Position 13 product specific lipoxygenase derived from soybean is commercially available from Sigma (Sigma, Product Number L7395) as a test reagent, while position 9 product specific lipoxygense is not commercially available.

The present inventors have found a α-ketol unsaturated fatty acid (hereinafter referred to as KODA) having a structure represented by the following Formula:

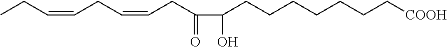 [SMILES: CC/C=C\C/C=C\CC(=O)C(O)CCCCCCCC(=O)O]

having a floral bud-formation promoting activity, a plant activation activity and a plant growth regulating activity comprising them, and KODA is known to be derived from α-linolenic acid by acting position 9 product specific lipoxygenase and allene oxide synthase (Japanese Unexamined Patent Publication (Kokai) No. 9-295908, Japanese Unexamined Patent Publication (Kokai) No. 11-29410, Japanese Unexamined Patent Publication (Kokai) No. 2001-131006, and Japanese Unexamined Patent Publication (Kokai) No. 2009-17829).

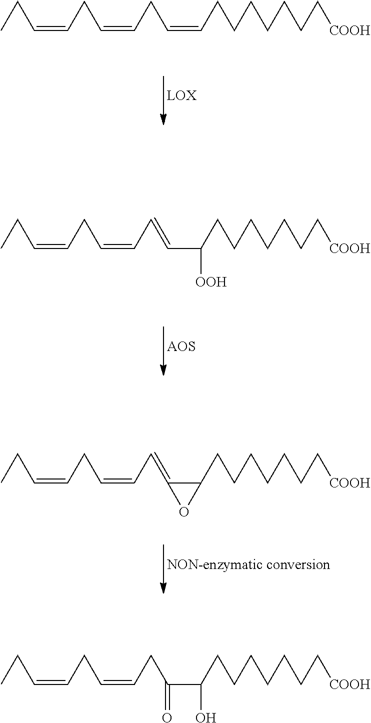

KODA was known to be present in a variety of plant species, and Lemna paucicostata that was subjected to stress was known to release KODA at a very high level (a few hundred-fold higher) compared to other plants (Japanese Unexamined Patent Publication (Kokai) No. 11-29410). Thus, it was estimated that in Lemna paucicostata position 9 product specific lipoxygenase which has higher activity than in other plants are present. However, cDNA of position 9 product specific lipoxygenase has not been isolated, and the sequence thereof has been unknown.

When KODA is to be produced by enzymatic synthesis /with α-linolenic acid or linoleic acid as the starting substance, position 9 product specific lipoxygenase is required. However, position 9 product specific lipoxygenase is not commercially available, and when cDNA of currently known position 9 product specific lipoxygenase was expressed in Escherichia coli, most of them are turned out to be insoluble, and thus it was difficult to obtain active protein at large quantities. In addition, even the extraction from a plant requires a lot of time and effort on obtaining and processing materials, and the activity of position 9 product specific lipoxygenases obtained to date was low. Therefore, there has been a need for establishing a technology to prepare highly active form of position 9-product specific lipoxygenase at large amount.

### CITATION LIST

### Patent Literature

PLT1: Japanese Unexamined Patent Publication (Kokai) No. 9-295908
PLT2: Japanese Unexamined Patent Publication (Kokai) No. 11-29410
PLT3: Japanese Unexamined Patent Publication (Kokai) No. 2001-131006
PLT4: Japanese Unexamined Patent Publication (Kokai) No. 2009-17829

### Non Patent Literature

NPL1: Uchiyama Mitsuru, et al., lipid peroxide and biological body, 23-26, 1985, Japan Scientific Societies Press
NPL2: Yamamoto Shouzo, Protein Nucleic acid Enzyme, 44, 1132-1138, 1999
NPL3: Grechkin et al., Eur. J. Biochem., 199, 451-457, 1991

### SUMMARY OF INVENTION

### Technical Problem

The problem to be solved by the present invention is to identify highly active form of position 9 product specific lipoxygenase gene useful for the expression, to isolate cDNA, and to provide an expression product of said lipoxygenase gene, thereby to provide a method of preparing KODA in high yield by using the lipoxygenase.

### Solution to Problem

In intensive research to solve the above problem, and to isolate new lipoxygenase cDNA from Lemna paucicostata, the present inventors conducted a screening of various strains of Lemna paucicostata for the ability of producing KODA in order to obtain the cDNA of lipoxygenase having a higher activity. Specifically 62 strains of Lemna paucicostata were subjected to the screening, thereby it was surprisingly found that Lemna paucicostata SH strain has a KODA producing activity 10-fold or more higher than the mean of other Lemna paucicostata strains.

The present inventors have isolated lipoxygenase cDNA from Lemna paucicostata SH strain and Lemna paucicostata 441 strain, which is used as a representative strain, expressed the cDNA in Escherichia coli as host cell, and examined the product specificity. As a result, it was revealed that these lipoxygenases were confirmed to have position 9-product specific lipoxigenase activity, and that the activity thereof was higher than that of rice position 9 product specific lipoxigenase, which has been conventionally used. Thus, the present inventors provide a novel position 9 product specific lipoxygenase gene derived from Lemna paucicostata and a protein encoded by said gene. Specifically, the present invention provides a gene consisting of a DNA having a base sequence selected from the group consisting of SEQ ID NOs: 1, 3 and 5, a gene consisting of a DNA substantially identical to said DNA, and proteins encoded by these genes. Furthermore, the present inventors provide an expression vector having the above DNA and a vector fragment bound to each other therein. Furthermore, the present invention also relates to a transformant of a microorganism, an animal cell or an insect cell transformed with said expression vector. The position 9 product specific lipoxigenase of the present invention can be obtained by culturing said transformant, and extracting and purifying the position 9 product specific lipoxigenase.

### Advantageous Effects of Invention

Position 9 product specific lipoxigenase of the present invention has a high activity compared to the conventionally used position 9 product specific lipoxigenase, and further maintains its high activity when expressed in Escherichia coli. Therefore, α-ketol unsaturated fatty acid such as KODA can be prepared efficiently through enzymatic synthesis which uses α-linolenic acid or linoleic acid, etc., as a starting material by using the position 9 product specific lipoxigenase.

### BRIEF DESCRIPTION OF DRAWINGS

FIG. 1 is a graph showing the amount of KODA produced in 62 strains of Lemna paucicostata.

FIG. 2A shows the sequence of the LOX gene of Lemna paucicostata SH strain.

FIG. 2B shows the continuation of the sequence of FIG. 2A.

FIG. 2C shows the continuation of the sequence of FIG. 2B.

FIG. 3A shows the sequence of LOX1 gene of Lemna paucicostata 441 strain.

FIG. 3B shows the continuation of the sequence of FIG. 3A.

FIG. 3C shows the continuation of the sequence of FIG. 3B.

FIG. 4A shows the sequence of LOX2 gene of Lemna paucicostata 441 strain.

FIG. 4B shows the continuation of the sequence of FIG. 4A.

FIG. 4C shows the continuation of the sequence of FIG. 4B

FIG. 5A is a graph showing the HPLC chromatogram of linolenic acid-9-hydroperoxide synthesized by using r9-LOX1.

FIG. 5B is a graph showing the HPLC chromatogram of linolenic acid-9-hydroperoxide synthesized by using SHLpLOX.

FIG. 5C is a graph showing the HPLC chromatogram of linolenic acid-9-hydroperoxide synthesized by using 441LpLOX1.

FIG. 5D is a graph showing the HPLC chromatogram of linolenic acid-9-hydroperoxide synthesized by using 441LpLOX2.

FIG. 6 is a graph comparing the lypoxygenase activity of SHLpLOX, 441 LpLOX1, 441LpLOX2 and r9-LOX1, which is used as a control lypoxygenase.

### DESCRIPTION OF EMBODIMENTS

The embodiments of the present invention are explained below. The present invention relates to a novel position 9 product specific lipoxygenase gene derived from Lemna paucicostata and a protein encoded by said gene. Specifically, the present invention relates to a gene consisting of a DNA having a base sequence selected from the group consisting of SEQ ID NO: 1, SEQ ID NO: 3 and SEQ ID NO: 5, and a DNA substantially identical to said DNA, and to a protein encoded by the gene. It is preferable that the base sequence is the sequence represented by SEQ ID NO: 1 or SEQ ID NO: 3. It is further preferable that the base sequence is the sequence represented by SEQ ID NO: 1. As used herein the term “substantially identical DNA” refers to a DNA that has at least 70% identity to the base sequence of the reference DNA. The identity may preferably be at least 80%, at least 90%, at least 95%, at least 97%, at least 98%, at least 99%, at least 99.5%, or at least 99.9%. Furthermore, “substantially identical DNA” also refers to a DNA that can hybridize under a high stringent condition to a DNA consisting of a base sequence complementary to the reference DNA. The “substantially identical DNA”, when transcribed and translated, has the same enzyme activity as that of a protein encoded by the reference DNA, i.e., the position 9 product specific lipoxygenase activity. Furthermore, the present invention relates to a DNA that encodes a protein consisting of an amino acid sequence selected from the group consisting of SEQ ID NO: 2, SEQ ID No:4 and SEQ ID NO:6, and a DNA that encodes a protein consisting of an amino acid sequence substantially identical to said amino acid sequence. As used herein, “a protein consisting of substantially identical amino acid” is explained in detail below. In addition, the present invention also relates to a DNA fragment encoding a protein which has lypoxygenase activity.

Hybridization can be carried out by a known method or a method based on said method, such as a method described in J. Sambrook et al., Molecular Cloning, 2nd., Cold Spring Harbor Laboratory Press, 1989. In this connection, a high stringent hybridization condition refers to, for example, a NaCl concentration of about 10-40 mM, preferably about 20 mM, and a temperature of about 50-70° C., preferably about 60-65° C.

The present invention also relates to a protein consisting of an amino acid sequence selected from the group consisting of SEQ ID NO: 2, SEQ ID NO:4, and SEQ ID NO: 6, and a protein consisting of an amino acid sequence substantially identical to said amino acid sequence. It is preferable that the amino acid sequence is the sequence of SEQ ID NO:2 or 4. It is most preferable that the amino acid sequence is the sequence of SEQ ID No:2. As used herein, “a protein consisting of a sequence substantially identical to amino acid” refers to a protein that consists of an amino acid sequence in which one or a few amino acids have been deleted, substituted or added in the amino acid sequence of the reference protein, and that has the activity of the reference protein, i.e., position 9 product specific lypoxygenase activity. Furthermore, “a protein comprising a substantially identical amino acid sequence” refers to a protein that comprises a sequence having an identity of at least 70% with the reference amino acid sequence and that has an lypoxygenase activity. The identity may preferably be at least 80%, at least 90%, at least 95%, at least 97%, at least 98%, at least 99%, at least 99.5%, or at least 99.9%. In addition, the present invention relates to a protein which is encoded by a DNA of the base sequence selected from the group consisting of SEQ ID No:1, SEQ ID NO:3, and SEQ ID No:5, or a DNA substantially identical to said DNA, and which has position 9 product specific lypoxygenase activity. In addition, the present invention also relates to a protein fragment which has lypoxygenase activity.

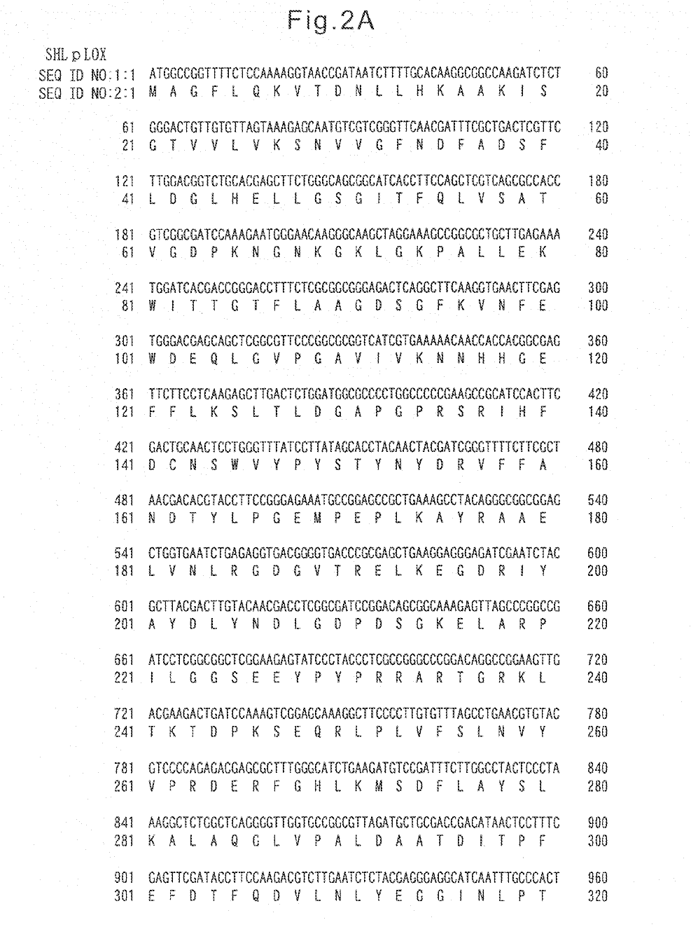

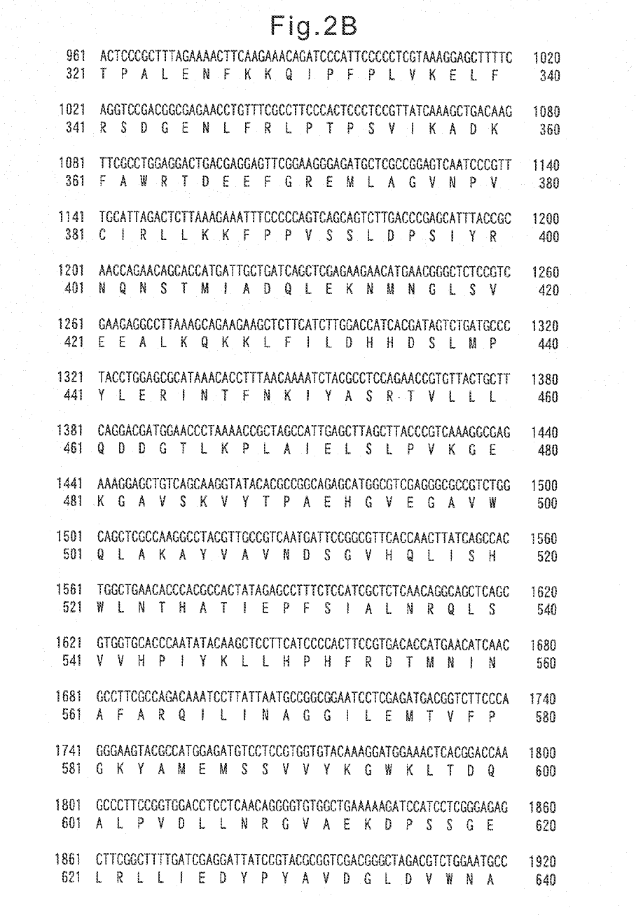

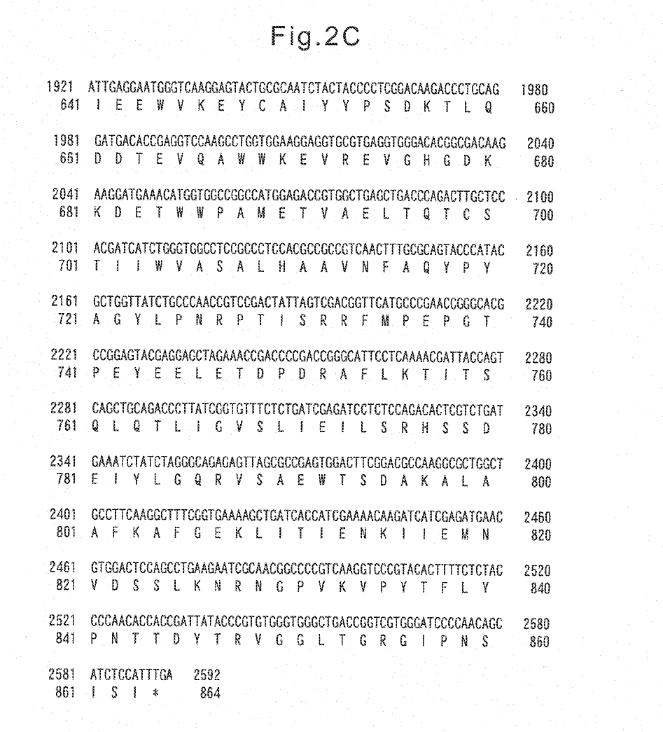

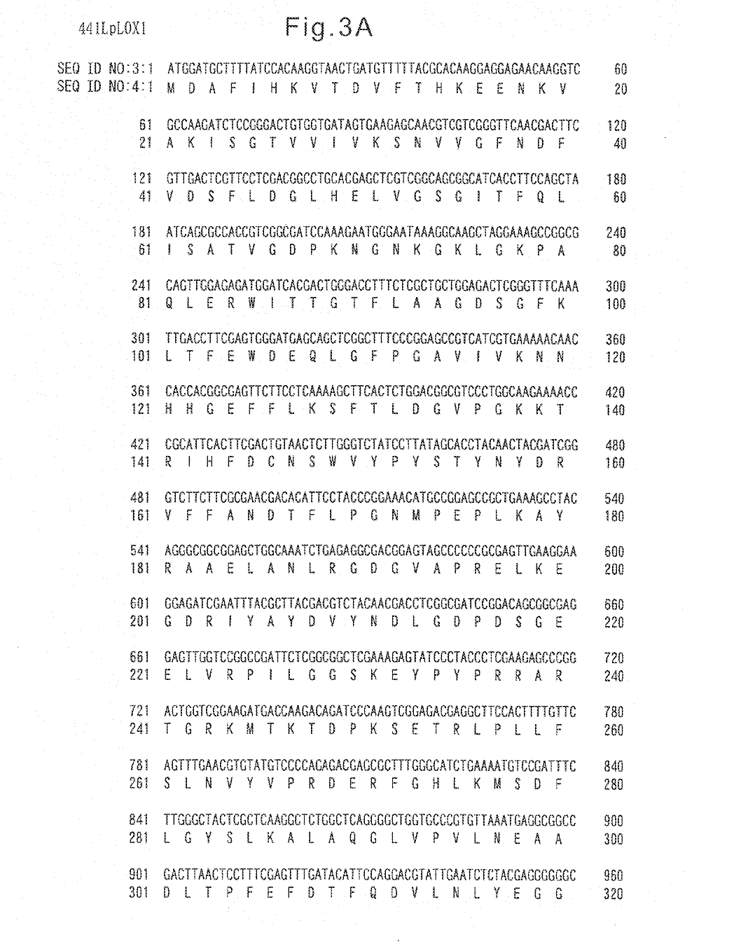

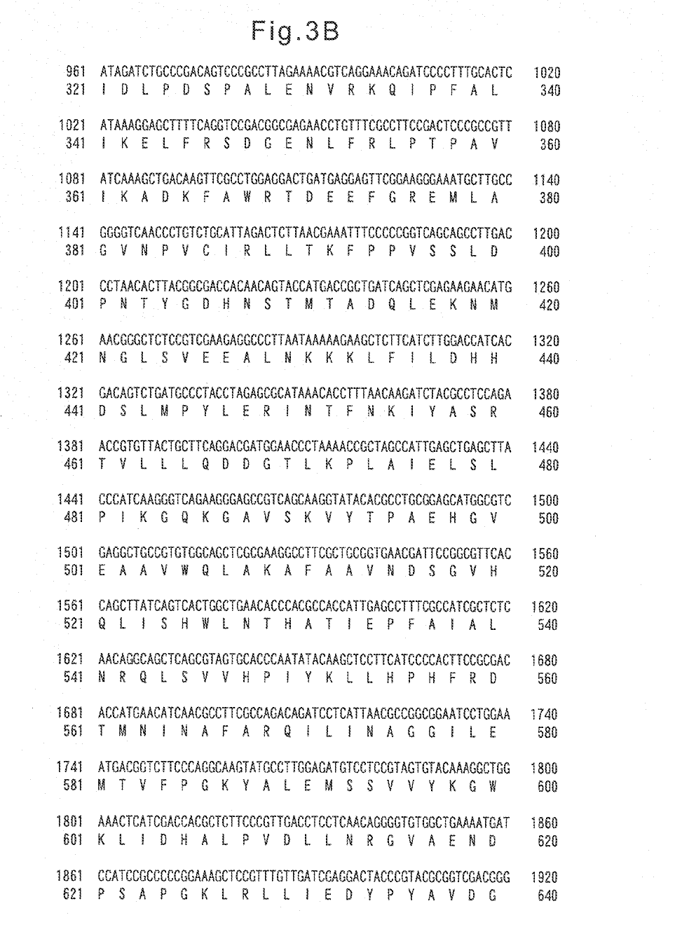

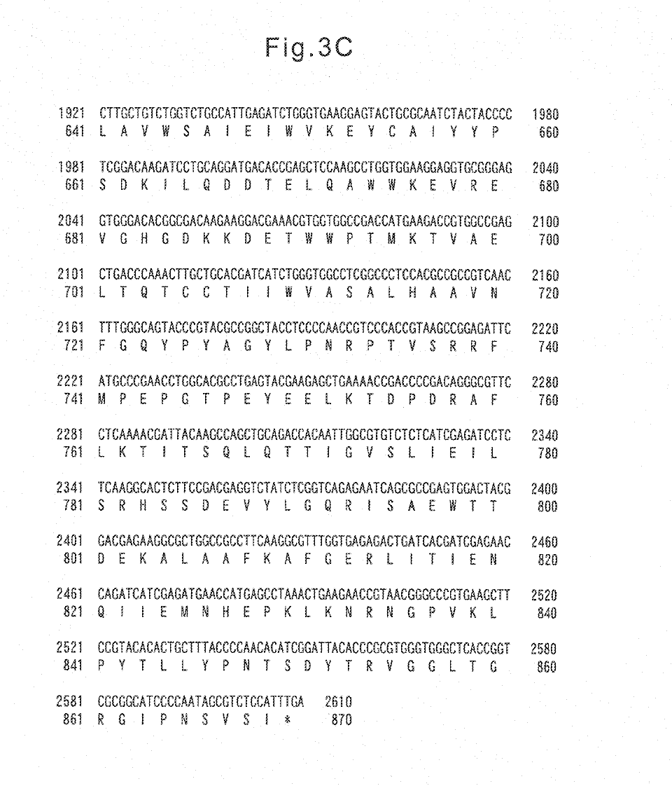

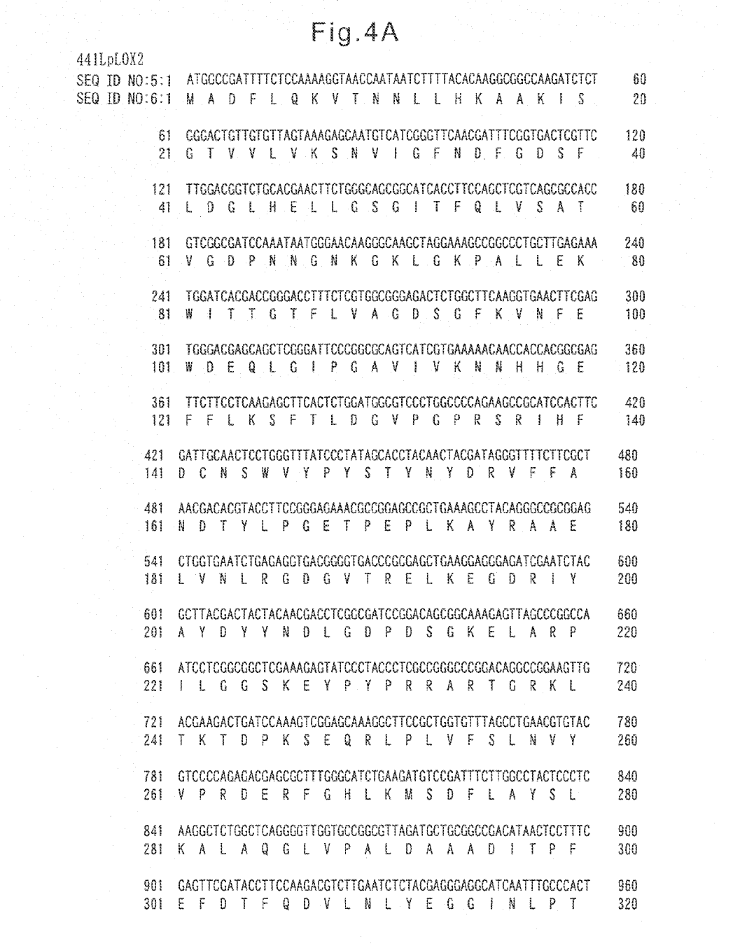

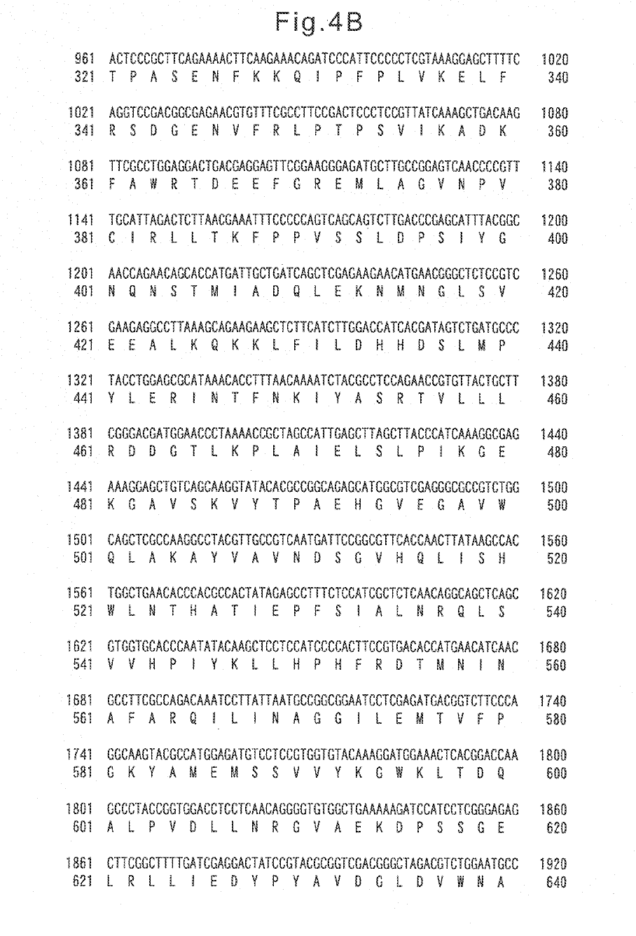

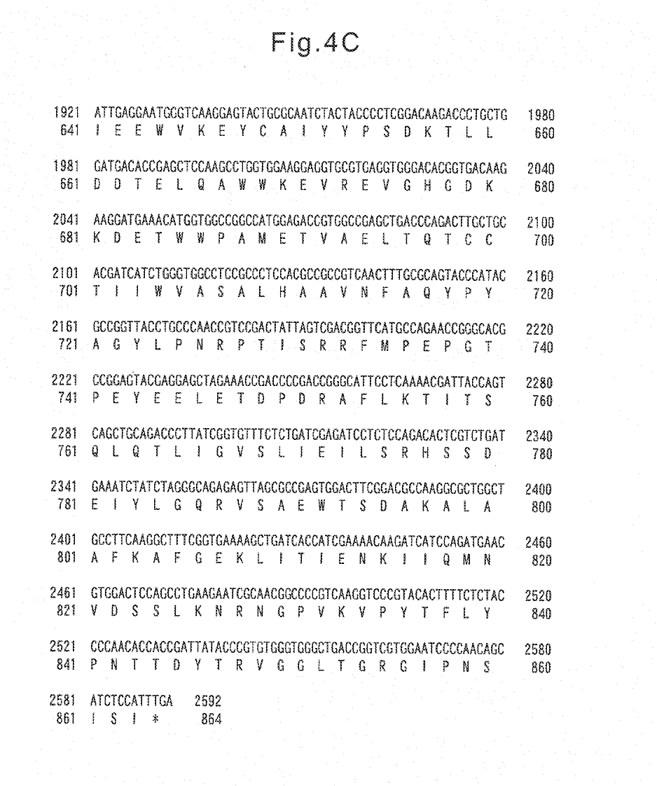

As used herein SEQ ID NO: 1 represents the base sequence of the position 9 product specific lypoxygenase derived from Lemna paucicostata SH strain, and SEQ ID NO: 2 represents the amino acid sequence of said lypoxygenase. On the other hand SEQ ID No:3 represents the base sequence of the position 9 product specific lypoxygenase 1 derived from Lemna paucicostata 441 strain, and SEQ ID NO: 4 represents the amino acid sequence of said lypoxygenase, SEQ ID No:5 represents the base sequence of the position 9 product specific lypoxygenase2 derived from the Lemna paucicostata 441 strain, and SEQ ID NO:6 represents the amino acid sequence of said lypoxygenase. These concrete sequences are shown in FIG. 2A-4C.

Position 9 product specific lypoxygenase activity means an enzymatic activity which uses an unsaturated fatty acid having carbon number of 18, such as linoleic acid as a substrate, and which introduces molecular oxygen as a hydroperoxide group into carbon at position 9 of the unsaturated fatty acid.

As used herein, position 9 product specific lypoxygenase or a protein having position 9 product specific lypoxygenase activity refers to one that is recognized to have a significant position 9 product specific lypoxygenase activity when determined by the activity determination method described below. For example, compared to the activity of position 9 product specific lypoxygenase having an amino acid sequence of SEQ ID NO: 2 or SEQ ID No:4, those having an activity of at least 10%, at least 50%, at least 70%, at least 80%, at least 90%, more preferably at least 95%, and furthermore 100% or more are encompassed.

The gene (cDNA) of the present invention can be obtained by, for example, the following procedure. From an amino acid sequence common to a plant-derived lypoxygenase, primers are designed, and RT-PCR is carried out to obtain a cDNA fragment. This fragment is labelled, as needed, with ³²P or digoxgenine, etc., and it is used in 5′-RACE or 3′-RACE, or screening is carried out in accordance with a common method by using cDNA library constructed in a standard method, thereby the full-length of lypoxygenase gene (cDNA) can be obtained. The determination of the base sequence can be conducted by a common method, such as the Sanger method and the Maxam Gilbert method.

In an alternative method, Lemna paucicostata-derived position 9 product specific lypoxygenase is purified using ammonium sulfate fractionation or various chromatographic methods, separated by SDS polyacrylamide gel electrophoresis, electrically transferred to a PVDF membrane etc., and excised a band detected with a dye such as Coomassie brilliant blue. Then, the N-terminal amino acid sequence is determined with a protein sequencer. Based on this sequence, primers are designed, and PCR is carried out according to the method mentioned above to obtain the full-length of the lypoxygenase gene gene (cDNA).

The isolated gene (cDNA) can be inserted into a plasmid, virus etc., by a commonly known method so as to prepare an expression vector, which is then introduced into the host cells such as a microorganism, or cultured cells of animal cells, plant cells, insect cells, etc. Then, the cells are allowed to express the gene, thereby the protein can be produced in large quantities. Furthermore, as the protein-expression system, a cell-free protein expression system, for example a system using a wheat germ extract, may be used.

As a vector, an Escherichia coli-derived plasmid such as pBR322, pBR325, pUC18 and pUC119, a Bacillus subtilis-derived plasmid such as pUB110, pTP5 and pC194, a yeast-derived plasmid such as pSH19 and pSH15, a bacteriophage such as λ phage, an animal virus such as retrovirus, vaccinia virus and baculovirus, and pA1-11, pXT1, pRC/CMV, pRC/RSV, pcDNAI/Neo etc., can be used. pET23d (Novagen), which is a commercially available vector, can be used in the present invention.

The expression vector comprising the gene of the present invention can be produced by ligating the gene of the present invention downstream to a promoter of a suitable vector via a suitable restriction site by a known method. The promoter to be used in the present invention may not be specifically limited as long as it is a suitable promoter corresponding to the host to be used for the expression of the gene. For example, when an animal cell is used as the host, SRα promoter, SV40 promoter, LTR promoter, CMV promoter, HSV-TK promoter, β-actin promoter etc. can be mentioned. When the host is a bacteria of the genus Escherichia such as Escherichia coli, trp promoter, lac promoter, recA promoter, λPL promoter, lpp promoter, T7 promoter etc. may preferably be used, when the host is a bacteria of the genus bacillus, SPO1 promoter, SPO2 promoter, pen promoter may preferably be used, and when the host is a yeast, PHO5 promoter, PGK promoter, GAP promoter, ADH promoter etc., may preferably be used. When the host is an insect, polyhedron promoter, P10 promoter etc., may be preferred. As desired, the expression vector may further contain an enhancer, a splicing signal, a polyA addition signal, a selection marker, SV40 replication origin, etc.

The host cell may be, but not limited to, a procaryotic cell or an eucaryotic cell that is conventionally used as the host for producing recombinant protein, and may include a mammal cell, an insect cell, a plant cell or a fungal cell, and preferably microorganisms such as bacteria or fungi. The term “fungus” is intended to encompass filamentous microorganisms and yeasts. Examples of suitable bacteria include Gram positive bacteria including genus Escherichia such as Escherichia coli, genus Bacillus such as Bacillus subtilis, and genus Streptomyces such as S. lividans, and Gram negative bacteria including genus Pseudomonas such as P. cepacia. The cells may be of fungi, i.e. yeast or filamentous cells. Yeast may be cells of genus Saccharomyces such as S. cereviceae. Filamentous host orgamisms may be, for example, Aspergillus strains such as A. niger. As insect cells, Sf cells, MG1 cells, BmN cells etc. may be mentioned. As animal cells, for example, monkey cell COS-7 cells, Vero cells, Chinese hamster cells CHO etc. may be mentioned. As used herein, Escherichia coli cells may specifically be preferred.

For the transformation of host cells, a known method such as the calcium chloride method, the calcium phosphate coprecipitation method, the DEAE dextran method, the lipofectin method, the protoplast polyethylene fusion method, the electroporation method etc. can be used, and may be selected as appropriate depending on the host cell to be used.

The protein of interest can be recovered by culturing the recombinant host cells obtained in a suitable culture medium, separating the cells from the culture liquid by a common procedure including, but not limited to, centrifugation or filtration, disrupting the cells as needed, precipitating the proteinaceous components of the supernatant or the filtrate by a salt such as ammonium sulfate, and then performing various chromatographic procedures such as ion exchange chromatography and an affinity chromatography.

If desirable, by ligating a suitable signal sequence for secretion in microorganisms or animals to upstream of a DNA encoding the protein of the present invention, said protein can be secreted and expressed in the culture medium. Such a DNA modified to a secretary type is useful since it facilitates the purification of the protein secreted into the culture medium. As examples of such a signal sequence, there can be mentioned the pel B signal (S. P. Lei et al., J. Bacteriology, 169: 4379-4383, 1987) in the case of Escherichia coli, a factor signal (A. J. Brake, Yeast Genetic Engineering, p269 Butterworth, 1989) in the case of yeast, the signal SG-1 of immunoglobulin (H. Maeda et al., Hum. Antibod. Hybridomas, 2: 124-134, 1991), C25 signal (W094/20632) etc. in the case of animal cells.

The lypoxygenase activity can be determined by the method generally used. For example, position 9 lypoxygenase is acted with a substrate, such as α-linolenic acid or linoleic acid, and then a method for detecting an increase of absorbance at 234 nm, which is resulted from the production of an enzymatic reaction production, hydroxyperoxide form, for detecting reduction of dissolved oxigen in reaction solution, which is resulted from the consumption of molecular oxygen, or directly detecting hydroxyperoxide form in HPLC can be used. In addition, the position 9-product specificity can be detected by using normal-phase HPLC, etc., with a silica gel column as described in a prior document (Yamamoto et al., Lipids, 15, 1-5, 1980).

The protein of the present invention may be useful as a reagent for use in the synthesis of various linoleic acid and linolenic acid derivatives. Specifically, the protein of the present invention is useful as an enzyme to be used when an α-ketol unsaturated fatty acid, specifically KODA, useful as a plant growth regulating agent, is enzymatically synthesized from linoleic acid or linolenic acid. Also, it may be estimated that by introducing the gene of the present invention into a plant, the metabolism of linoleic acid or linolenic acid present in plant by position 9 product specific lypoxygenase can be controlled.

The present invention will now be explained more specifically with reference to examples below. However, these examples do not limit the technical scope of the present invention in any way.

### EXAMPLES

### Example 1

### Screening of a High KODA-Producing Lemna Paucicostata Strain

62 types of Lemna paucicostata harvested from different places were prepared and were passaged in the ½-diluted Hutner's medium under continuous illumination from a 24-25° C. daylight fluorescent light. The ½-diluted Hutner's medium had the following ingredients:

| | | |
|---|---|---|
| Sucrose | 10 | g/l |
| K₂HPO₄ | 200 | mg/l |
| NH₄NO₃ | 100 | mg/l |
| EDTA free acid | 250 | mg/l |
| Ca(NO₃)•4H₂O | 176 | mg/l |
| MgSO₄•7H₂O | 250 | mg/l |
| FeSO₄•7H₂O | 12.4 | mg/l |
| MnCl₂•4H₂O | 8.92 | mg/l |
| ZnSO₄•7H₂O | 32.8 | mg/l |
| Na₂MoO₄•2H₂O | 12.6 | mg/l |
| H₃BO₃ | 7.1 | mg/l |
| Co(NO₃)•6H₂O | 0.1 | mg/l |
| CuSO₄•5H₂O | 1.97 | mg/l |

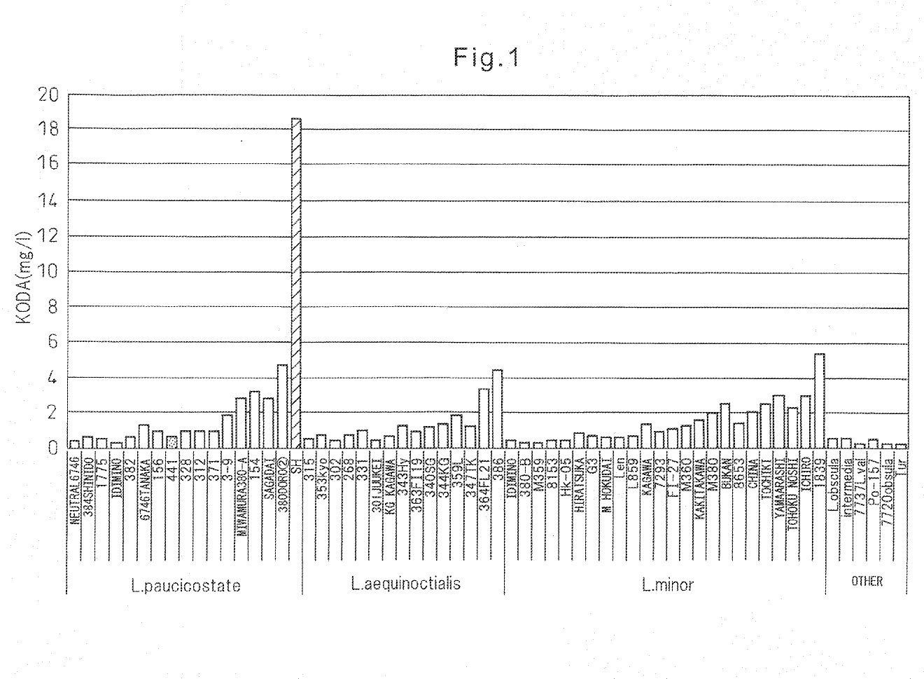

The grown Lemna paucicostata was unfolded on a filter paper, and after allowing to stand for 2 hours, it was immersed in water for 1 hour. For the water, the concentration of KODA was analyzed using high performance liquid chromatography (HPLC; column: TYPE UG120 5 μm, SIZE: 4.6 mm I.D.×250 mm; guard filter: INERTSTL 4.6 mm×50 mm; eluent: 50% acetonitrile+0.1% trifluoroacetic acid; condition: absorption wavelength 210 λ (nm), flow rate: 1 ml/min, column temperature: 40° C.). The mean of the amount produced of KODA in all Lemna paucicostata strains was 4.97 μM. Among them, Lemna paucicostata SH strain produced 60.2 μM of KODA, which is about 12-times as much as the mean amount of KODA produced, giving a very high production amount (FIG. 1).

### Example 2

### Cloning of Lipoxygenase Derived from Lemna Paucicostata SH Strain and Measurement of the Activity Thereof

From Lemna paucicostata SH strain and Lemna paucicostata 441 strain, total RNA was extracted by using the RNeasy Plant Mini Kit (QIAGEN), and then cDNA was synthesized by using 1.8 μg of total RNA of each strain of Lemna paucicostata as the template in LongRange 2 Step RT-PCR Kit (QIAGEN).

Then, degenerate PCR (PCR condition: initial denaturation at 94° C. for 3 minutes; a cycle comprising 94° C. for 0.5 minute, 47° C. for 0.5 minute, and 72° C. for 1.3 minute is carried out for 39 times) was carried out by using the cDNA as the template, and using the following degenerate primers (LpDPf, LpDPr) to obtain a partial sequence of 9-lipoxygenase of interest.

(SEQ ID NO: 7)
LpDPf: 5′-GCITGGMGIAGIGAYGARGARTTY-3′
(SEQ ID NO: 8)
LpDPr: 5′-GCRTAIGGRTAYTGICCRAARTT-3′

wherein, I represents inosine.

In order to design primers for cloning Lemna paucicostata lypoxygenase gene (cDNA), these degenerate primers were constructed from a region wherein high homology is present in amino acid sequences of lypoxygenase derived from plants which had been cloned already (soybean, potato, tomato, barley, rice).

One sequence of product and two sequences of products having about 1.0 kb, which are respectively corresponds to the sentral region of lypoxygenase cDNA from 441 strain are obtained, are obtained, and determined for the base sequences. Based on the obtained sequence information, BLAST search was carried. As a result, these three sequences exhibited a high homology with LOX derived from a plurality of known plants (75% with Corylus avellana, 74% with Actinidia deliciosa, 75% with Solanum tuberosum, 76% with Oryza sativa, 74% with Nicotiana tabacum, 75% with Cucumis sativus, 73% with Arabidopsis thaliana, etc.).

Based on this sequence information, primers for the following 3′ or 5′ RACE method (Rapid Amplification of cDNA end) were constructed:

3′-Race primer
(SEQ ID NO: 9)

| | |
|---|---|
| SH-3′-TP: | 5′-AGCTCTTCATCTTGGACC-3′ |

(SEQ ID NO: 10)

| | |
|---|---|
| 441-1-3′-TP: | 5′-AAGCTTCTTCATCCCCACTTCC-3′ |

(SEQ ID NO: 11)

| | |
|---|---|
| 441-2-3′-TP: | 5′-AAGCTCCTTCATCCCCACTTCC-3′ |
| 5′-Race primer | |

(SEQ ID NO: 12)

| | |
|---|---|
| SH-5′-TP: | 5′-TTTCATCCTTCTTGTCGC-3′ |

(SEQ ID NO: 13)

| | |
|---|---|
| 441-1-5′-TP: | 5′-GCTTGTATATTGGGTGCAC-3′ |

(SEQ ID NO: 14)

| | |
|---|---|
| 441-2-5′-TP: | 5′-AAGCAGTAACACGGTTCTGGAGG-3′ |

### 3′-RACE

2 μg of total RNA obtained from SH strain or 441 strain was used as a template so as to synthesize cDNA by using Cap Fishing™ target full-length cDNA cloning Kit (Seegene). This cDNA is used as a template solution, and then PCR is each carried out by using adapter primer (3′-RACE primer) contained in a kit and SH-3′-TP, 441-1-3′-TP, or 441-2-3′-TP.

### 5′-RACE

2 μg of total RNA obtained from SH strain or 441 strain was used as a template so as to synthesize cDNA by using Cap Fishing™ target full-length cDNA cloning Kit (Seegene). This cDNA is used as a template solution, and then PCR is each carried out by using adapter primer (5′-RACE primer) contained in a kit and SH-5′-TP, 441-1-5′-TP, or 441-5′-TP.

The sequence of each product was determined, and then the products were connected into one sequence by using an appropriate restriction site so as to obtain whole length cDNA, which is then sub-cloned into pTAC-2 vector (BioDynamics).

Lypoxygenase obtained from Lemna paucicostata SH strain is named as SHLpLOX, and Lypoxygenase obtained from Lemna paucicostata 441 strain is named 441LpLOX1 and 441LpLOX2.

Regarding three kinds of novel LOXcDNAs derived from Lemna paucicostata 441 strain and SH strain, a DNA fragment comprising full length of translation region consisting of starting codon to terminate codon was obtained by using PCR with primers having NcoI clevage domain at 5′ end, and NotI cleavage domain at 3′ end, and then inserted into NcoI/NotI sites of commercially available expression vector, pET23d(Novagen), thereby plasmide pET23d/SHLpLOX, pET23d/441LpLOX1 and pET23d/441LpLOX2 were obtained. These plasmide was transformed into Escherichia coli BL21 (DE3) strain (Novagen). Then, the microorganism was inoculated into LB medium containing 10mg/L ampicilin, and then shake-cultured at 37 for overnight. 100 μl of the culture medium was added to 10 ml of LB medium containing 10 mg/L ampicilin, and then shake-cultured at 37° C. for 5 hours. the fungus body was recovered by centrifuging from the culture medium, and then transferred to TB medium containing 0.1 mM isopropyl-β-D-thiogalactopyranocid (Wako), 100 mg/l of ampicilin and cultured for 40 hours at 15° C. so as to induce the expression of lypoxygenase. After the culture, the fungus body was collected by centrifugation (wet weight 0.4 g), and was dissolved in 10 ml of BugBuster™ (Novagen). Then centrifugation (9000 rpm, 5 minutes, 4° C.) was carried out so as to collect supernatant (10 ml) as a enzyme solution.

As a control enzyme solution of position 9 specific lypoxygenase, an enzyme solution obtained by expressing and extracting lypoxygenase as described above from Escherichia coli BL21 (DE3) transformed by a plasmid, wherein r9-LOX1, which is position 9 specific lypoxygenase derived from rice germ, was inserted into NcoI/NotI site of pET23d, was used.

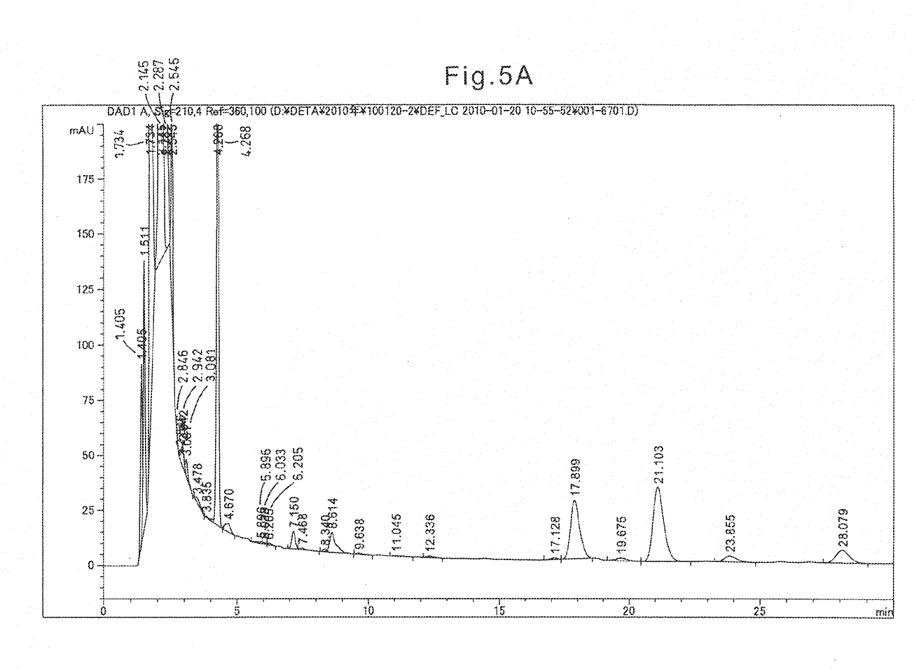

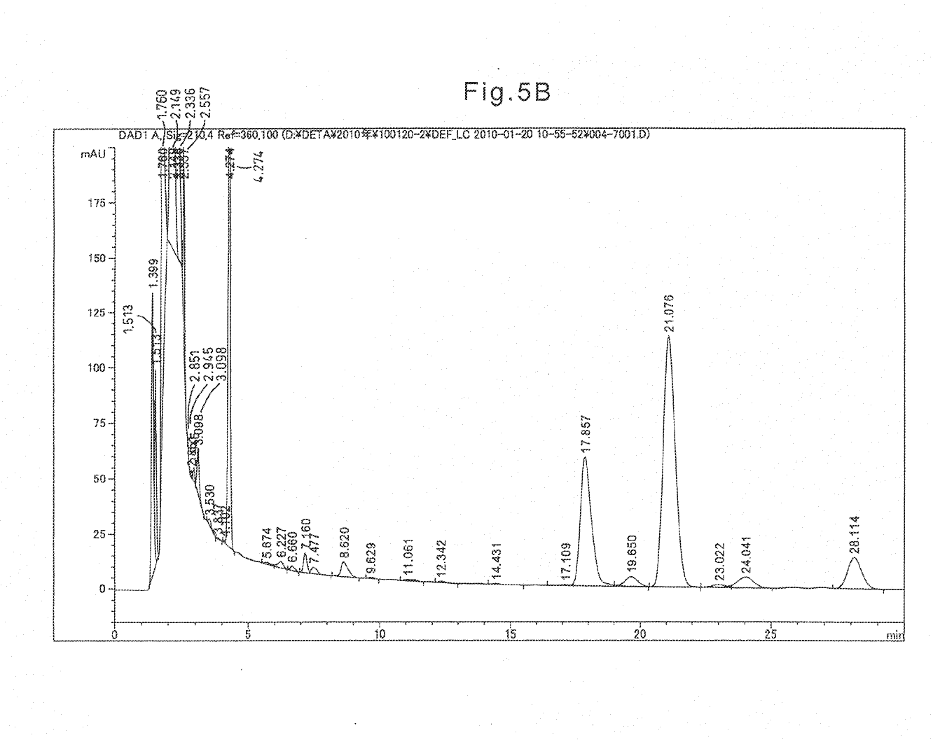

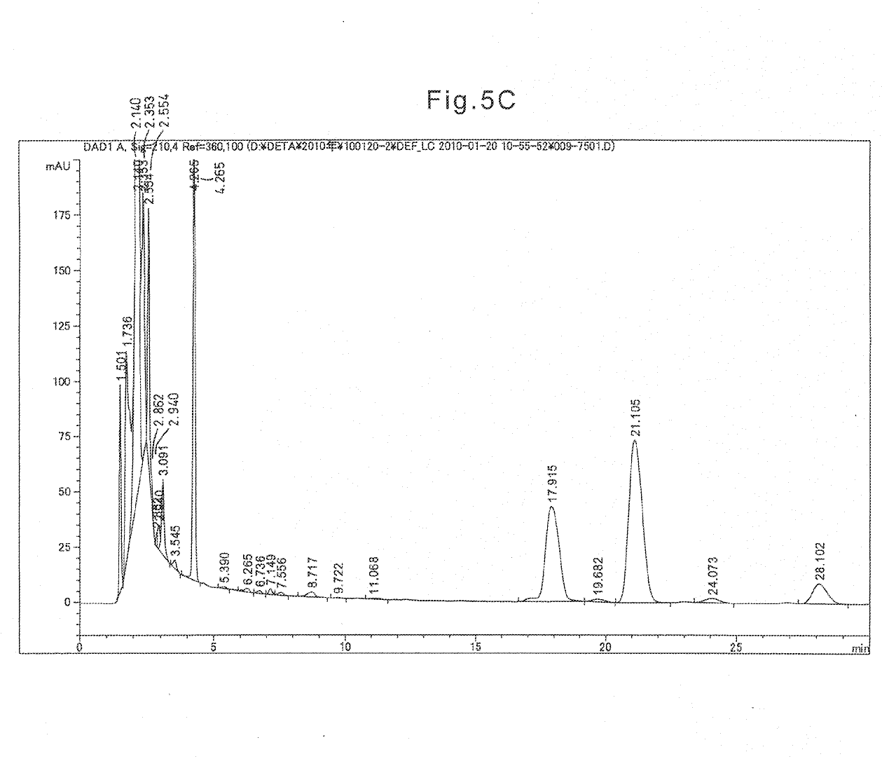

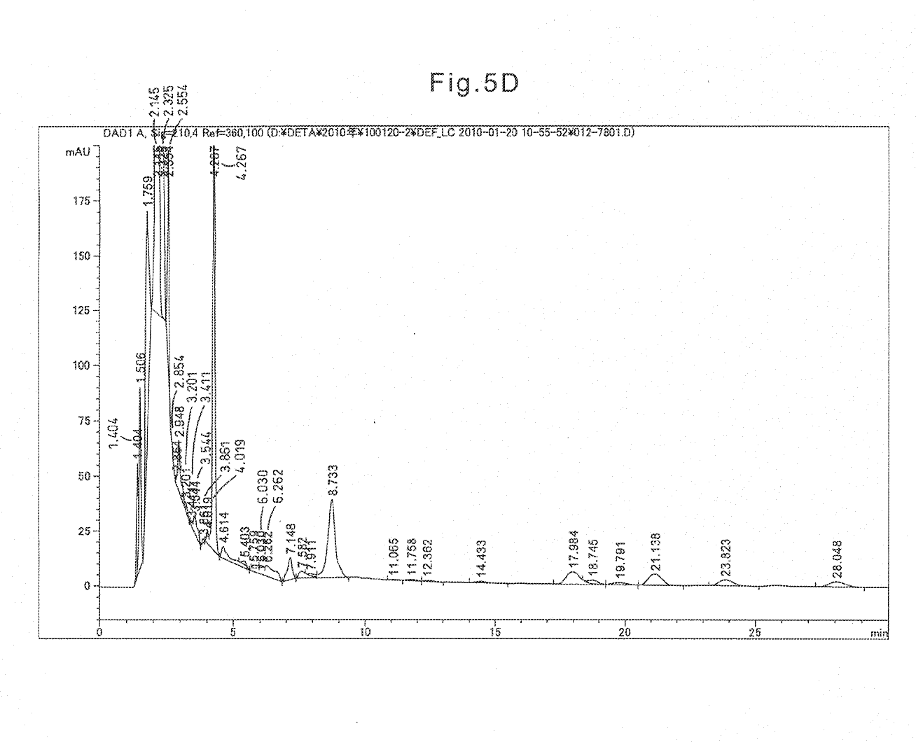

The activity of lypoxygenase in the enzyme solutions of SHLpLOX, 441LpLOX1, 441LpLOX2 and r9-LpLOX1 which was used as a control, was determined. 25 μl of 5 mM α-lynolenic acid solution (including 0.05% Tween80), 10 μl of 0.2M phosphate Na Buffer (pH7.0), 5 μl of distilled water were added to 1.5 ml of tube, and then 10 μl of the enzyme solution was added to the tube, and allowed to react for 30 minutes. After the reaction was over, reactant was subjected to HPLC (reverse phase; Column: Capsule pack C-18 UG120 (4.6×250 mm, Shiseido), Column temperature: 40° C., Mobile phase: 50% acetnitril (0.02%TFA), Flow rate 1 ml/min, Detection: 210 nm) so as to detect the site specificity of produced lynolenic acid hydroperoxide and the production amount. The result of HPLC was shown in FIG. 5A-D. As shown in a HPLC chart, a peak of linolenic acid-9-hydroxyperoxide (21.1 min) was confirmed in each chart of SHLpLOX, 441LpLOX1, 441pLOX2, which revealed that the enzymes have position 9 product specific lypoxygenase activity.

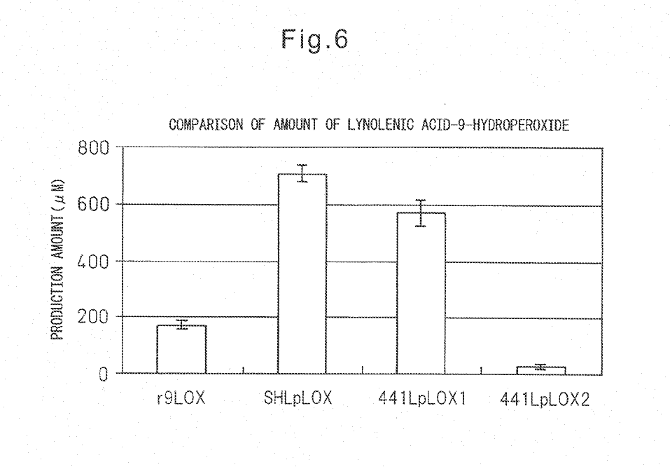

The amount of lynolenic acid-9-hydroperoxide form was compared based on the results of HPLC. The result of the comparison was shown in FIG. 6. The Novel LOX derived from Lemna paucicostata SH strain (SHLpLOX) and the novel LOX derived from Lemna paucicostata 441 strain (441LpLOX1) exert far higher activity of position 9-product specific lypoxygenase than that of r9-LOX1, which has been known to have strongest activity among the known position 9-product specific lyopxygenase.

Among the novel LOXs derived from Lemna paucicostata, SHLpLOX that had the highest production amount of linolenic acid-9-hydroperoxide was subjected to kinetic analysis. Using a reaction mixture comprising 40 mM phosphate buffer (pH 6.0) and 0.1% Tween 80, the analysis was carried out at a temperature of 25° C. The substrate α-linolenic acid was tested at the substrate concentration of 10-100 μM. 100 μl of the reaction mixture was added into a cuvette, and absorbance at 234 nm was scanned over time for 10 minutes at an interval of 15 seconds using the SmartSpec Plus Spectrophotometer (Bio-Rad). The amount of the reaction product was calculated from the A₂₃₄ determined (e=25,000). Kinetic parameters were determined using the Hanes-Woolf plot ([S]/v versus [S] plot). As shown in Table 1, the result shows that the Kₘ value which is an affinity parameter for substrate is lower in SHLpLOX than in r9-LOX1, thereby indicating that SHLpLOX had a high affinity for the substrate α-linolenic acid. The maximum reaction velocity Vₘₐₓ is almost comparable between r9-LOX and SHLpLOX, while the k_{cat} value which is the number of reactions per unit time was higher in SHLpLOX than in r9-LOX1. The k_{cat}/kₘ value which is an index of enzyme activity was about 1.6-fold higher in SHLpLOX than in r9-LOX1. This revealed that the novel 9-LOX derived from Lemna paucicostata SH strain is a very highly active 9-LOX

TABLE 1
Kinetic parameters of SHLpLOX and r9-LOX1

| | Kₘ | Vₘₐₓ | k_{cat} | K_{cat}/Kₘ |
|---|---|---|---|---|
| | (μM) | (μmol min⁻¹) | (min⁻¹) | (×10⁵M⁻¹min⁻¹) |
| LpLOX-SH | 19.2 ± 4.1 | 3.0 ± 0.1 | 768.2 ± 32.3 | 41.4 ± 10.0 |
| r9-LOX1 | 22.9 ± 1.6 | 2.8 ± 0.6 | 614.1 ± 124.6 | 26.7 ± 4.2 |

1. A gene consisting of a DNA selected from the group consisting of the following (a) to (e):
(a) a DNA consisting of a base sequence of SEQ ID NO: 1;
(b) a DNA that encodes a protein consisting of an amino acid sequence of SEQ ID NO: 2;
(c) a DNA encoding a protein that consists of an amino acid sequence having at least 99% identity to the amino acid sequence of SEQ ID No:2, and that has position 9 product specific lypoxygenase activity;
(d) a DNA encoding a protein that consists of an amino acid sequence in which one or a few amino acids have been deleted, substituted or added in the amino acid sequence of SEQ ID NO: 2, and that has position 9 product specific lypoxygenase activity;
(e) a DNA that hybridizes under a high stringent condition to a DNA comprising a base sequence complementary to a DNA comprising a base sequence represented by SEQ ID NO: 1, and that encodes a protein having position 9 product specific lypoxygenase activity.

2. An expression vector comprising the DNA according to claim 1 and a vector fragment bound to each other therein.

3. A transformant wherein the expression vector according to claim 2 has been introduced into the host microorganism, animal cell or plant cell.

4. A method of producing position 9 product specific lypoxygenase, characterized in that comprising culturing the transformant according to claim 3.

5. A protein selected from the group consisting of the following (a) to (d):
(a) a protein consisting of an amino acid sequence represented by SEQ ID NO: 2;
(b) a protein that consists of an amino acid sequence in which one or a few amino acids have been deleted, substituted or added in an amino acid sequence represented by SEQ ID NO: 2, and that has position 9 product specific lypoxygenase activity;
(c) a protein that consists of an amino acid sequence having an identity of at least 99% with amino acid sequence of SEQ ID NO:2, and has position 9 product specific lypoxygenase activity; and
(d) a protein that is encoded by a polynucleotide capable of hybridizing under a high stringent condition to a DNA comprising a base sequence complementary to a base sequence represented by SEQ ID NO: 1 and that has an position 9 product specific lypoxygenase activity.
